(19) United States
(12) Patent Application Publication
(10) Pub. No.: US 2020/0057055 A1
(43) Pub. Date: Feb. 20, 2020

(54) NONSPECIFIC REACTION INHIBITOR

(71) Applicant: TANAKA KIKINZOKU KOGYO K.K., Tokyo (JP)

(72) Inventors: Keita SUZUKI, Kanagawa (JP); Hisahiko IWAMOTO, Kanagawa (JP)

(73) Assignee: TANAKA KIKINZOKU KOGYO K.K., Tokyo (JP)

(21) Appl. No.: 16/609,967

(22) PCT Filed: May 2, 2018
(86) PCT No.: PCT/JP2018/017551

§ 371 Date: Oct. 31, 2019

(30) Foreign Application Priority Data
May 2, 2017 (JP) 2017-091858

(51) Int. Cl. G01N 33/53; C07K 16/42; G01N 33/543

(52) U.S. Cl. CPC G01N 33/5306; G01N 33/543; C07K 16/4283

(57) ABSTRACT

The invention relates to a nonspecific reaction inhibitor for an immunoassay containing an anti-mammal-derived IgM antibody in which, in ELISA assay, a ratio A2/A1 of an absorbance A2 which is obtained when a reaction of the anti-mammal-derived IgM antibody with cat IgM is carried out to an absorbance A1 which is obtained when a reaction of the anti-mammal-derived IgM antibody with dog IgM is carried out is 0.1 or more and 1.5 or less and in which, in ELISA assay, a ratio A3/A1 of an absorbance A3 which is obtained when a reaction of the anti-mammal-derived IgM antibody with human IgM is carried out to the absorbance A1 which is obtained when the reaction of the anti-mammal-derived IgM antibody with dog IgM is carried out is 0.5 or more, which can sufficiently inhibit a nonspecific reaction.

### TECHNICAL FIELD

The present invention relates to a nonspecific reaction inhibitor for an immunoassay, to an immunochromatographic test strip and an immunochromatographic test kit which contain the nonspecific reaction inhibitor and to an immunoassay in which the immunoreaction is conducted in the presence of the nonspecific reaction inhibitor.

### BACKGROUND ART

Immunoassays using an antigen-antibody reaction are widely used for clinical examinations because a minor component can be measured specifically with high sensitivity. Examples of known immunoassays are enzyme immunoassay (for example, ELISA), agglutination assay, immunochromatography, radioimmunoassay, nephelometry and the like.

An antigen-antibody reaction is a highly specific binding reaction that is caused because the antigen-binding site of an antibody induced for an antigenic determinant is highly complementary to the antigenic determinant. In an immunoassay using the antigen-antibody reaction, however, a nonspecific reaction other than a targeted proper and specific antigen-antibody reaction occurs, and the reliability of the measured values is often reduced.

One of the causes for such a phenomenon is that an analyte contains a component which binds to an antibody used for the immunoassay although the component is not a substance to be detected (an antigen) (the component is called a nonspecific factor below). When the analyte contains a nonspecific factor, obtained measurement results indicate presence of the substance to be detected even though the substance to be detected is not actually contained.

Such nonspecific factor is a substance which binds not only to an antibody which specifically reacts with the substance to be detected but also to an antibody which does not react with the substance to be detected. Known nonspecific factors are heterophile antibodies and rheumatoid factors.

Heterophile antibodies are antibodies to an antibody which is derived from an animal kind other than human and contained in human blood or the like and examples thereof include human anti-mouse antibody (HAMA) as well as human anti-goat antibody (HAGA), human anti-sheep antibody (HASA), human anti-rabbit antibody (HARA) and the like.

Rheumatoid factors are antibodies to human antibodies contained in human blood or the like and are autoantibodies that are often found in rheumatoid arthritis patients.

It is said that, in addition to the heterophile antibodies and the rheumatoid factors, there are many nonspecific factors whose components are not clear yet.

The presence of a nonspecific factor in the analyte is a serious problem which reduces the advantage of immunoassays, namely, the ability of specifically measuring a minor component with high sensitivity, and which leads to a false diagnosis of a disease of a patient or the like in the field of clinical examinations. Therefore, various attempts have been made to inhibit nonspecific reactions due to nonspecific factors and to obtain a correct measurement value.

Patent Literature 1 discloses an immunological assay which can inhibit a nonspecific reaction due to a nonspecific factor and which can accurately measure an antigen by adding, to the immunoassay system, an anti-human IgM antibody or an anti-human IgG antibody which reacts with a natural antibody of IgM type or IgG type which is a nonspecific factor contained in a sample.

Patent Literature 2 describes an anti-human immunoglobulin M monoclonal antibody which reacts specifically with human immunoglobulin M and which can cause immunoagglutination with human immunoglobulin M based on antigen-antibody reaction in solution state. Patent Literature 2 further discloses an immunological assay which can inhibit a nonspecific reaction using the antibody.

Patent Literature 3 describes a nonspecific reaction inhibitor containing an anti-human rheumatoid factor (IgM type) mouse monoclonal antibody (IgG type) and an immunological assay using the inhibitor and discloses that a nonspecific reaction due to a heterophile antibody could be inhibited in comparison with a heterophilic blocking reagent HBR.

### CITATION LIST

### Patent Literature

Patent Literature 1: JP-A-H11-287801

Patent Literature 2: JP-A-2011-27751

Patent Literature 3: JP-A-2016-65795

### SUMMARY OF INVENTION

### Technical Problem

However, although the conventional methods exhibit some effects of inhibiting a nonspecific reaction in immunoassays, the effects are not sufficient, and there is still room for improvement. Moreover, depending on the analyte, the effects of the conventionally used nonspecific reaction inhibitors are hardly obtained, and not a few nonspecific reaction inhibitors cannot inhibit a nonspecific reaction due to a nonspecific factor. Therefore, the nonspecific reaction inhibitors are not entirely satisfactory in practical applications.

Accordingly, an object of the invention is to provide a nonspecific reaction inhibitor which can sufficiently inhibit a nonspecific reaction due to a nonspecific factor even with an analyte in which the influence of the nonspecific factor such as a heterophile antibody could not be inhibited sufficiently with the conventionally used nonspecific reaction inhibitors.

### Solution to Problem

In the Patent Literatures, antibodies which specifically react with human-derived immunoglobulins such as human IgM and human IgG are used for methods for inhibiting a nonspecific reaction due to a nonspecific factor. This is because it has been believed that, when a nonspecific reaction in an immunoassay is to be inhibited, an antibody which reacts with a human-derived nonspecific factor should be used to remove the human-derived nonspecific factor. In an immunoassay using a human-derived analyte as in such a case, a nonspecific reaction inhibitor which specifically reacts with a human-derived nonspecific factor, namely a nonspecific reaction inhibitor which does not have cross-reactivity with factors other than the human-derived nonspecific factor, has been generally used.

The present inventors have focused on how an antibody which shows reactivity not only with a human-derived immunoglobulin but also with an immunoglobulin derived from an animal kind other than human would influence the inhibition of a nonspecific reaction and have conducted intensive studies. As a result, surprisingly, it has been found that a nonspecific reaction can be inhibited significantly when an immunoassay is conducted using an antibody which shows certain reactivities with dog-derived IgM and cat-derived IgM as well as with human-derived IgM. The invention has been thus completed.

Therefore, the present invention is described below.

[1] A nonspecific reaction inhibitor for an immunoassay, containing an anti-mammal-derived IgM antibody,

in which, in ELISA assay, a ratio (A2/A1) of an absorbance A2 which is obtained when a reaction of the anti-mammal-derived IgM antibody with cat IgM is carried out to an absorbance A1 which is obtained when a reaction of the anti-mammal-derived IgM antibody with dog IgM is carried out is 0.1 or more and 1.5 or less and in which, in ELISA assay, a ratio (A3/A1) of an absorbance A3 which is obtained when a reaction of the anti-mammal-derived IgM antibody with human IgM is carried out to the absorbance A1 which is obtained when the reaction of the anti-mammal-derived IgM antibody with dog IgM is carried out is 0.5 or more.

[2] The nonspecific reaction inhibitor according to the above 1, wherein the ratio A3/A1 is 2.5 or less.
[3] The nonspecific reaction inhibitor according to the above 1 or 2, wherein the anti-mammal-derived IgM antibody is an anti-human IgM antibody, an anti-dog IgM antibody or an anti-cat IgM antibody.
[4] The nonspecific reaction inhibitor according to any one of the above 1 to 3, wherein the anti-mammal-derived IgM antibody content is 0.5 μg or more and 20 μg or less.
[5] The nonspecific reaction inhibitor according to any one of the above 1 to 4, wherein the immunoassay is immunochromatography.
[6] An immunochromatographic test strip containing the nonspecific reaction inhibitor according to any one of the above 1 to 5.
[7] An immunochromatographic test kit containing the nonspecific reaction inhibitor according to any one of the above 1 to 5.
[8] An immunoassay for specifically detecting a substance to be detected in an analyte, wherein the immunoreaction is conducted in the presence of the nonspecific reaction inhibitor according to any one of the above 1 to 5.
[9] The immunoassay according to the above 8 which is enzyme immunoassay, agglutination assay or immunochromatography.
[10] The immunoassay according to the above 8 which is immunochromatography.

### Effects of Invention

When the nonspecific reaction inhibitor of the invention is used, a nonspecific reaction in an immunoassay can be inhibited sufficiently.

### BRIEF DESCRIPTION OF DRAWINGS

FIG. 1 is a figure showing an embodiment of an immunochromatographic test strip containing the nonspecific reaction inhibitor of the invention.

FIG. 2 is a graph showing the measurement results of the immunochromatography of Test Example 2.

FIG. 3 is a graph showing the measurement results of the immunochromatography of Test Example 3.

FIG. 4 is a graph showing the measurement results of the immunochromatography of Test Example 4.

### DESCRIPTION OF EMBODIMENTS

Although the invention is explained in detail below, the invention is not limited to the embodiments below and can be carried out with appropriate modifications within the scope of the object of the invention.

The nonspecific reaction inhibitor of the invention contains an anti-mammal-derived IgM antibody in which, in ELISA assay, a ratio (A2/A1) of an absorbance A2 which is obtained when a reaction of the anti-mammal-derived IgM antibody with cat IgM is carried out to an absorbance A1 which is obtained when a reaction of the anti-mammal-derived IgM antibody with dog IgM is carried out is 0.1 or more and 1.5 or less and in which, in ELISA assay, a ratio (A3/A1) of an absorbance A3 which is obtained when a reaction of the anti-mammal-derived IgM antibody with human IgM is carried out to the absorbance A1 which is obtained when the reaction of the anti-mammal-derived IgM antibody with dog IgM is carried out is 0.5 or more.

Here, the “dog IgM” means an immunoglobulin of IgM type derived from dog, and the “cat IgM” means an immunoglobulin of IgM type derived from cat. The “human IgM” means an immunoglobulin of IgM type derived from human. Moreover, the “anti-mammal-derived IgM antibody” means an antibody which is obtained using IgM derived from a mammal as an immunogen.

The nonspecific reaction inhibitor of the invention contains an anti-mammal-derived IgM antibody showing reactivities with dog IgM, cat IgM and human IgM indicated by the certain absorbance ratios and thus can sufficiently inhibit a nonspecific reaction in an immunoassay. A reason why the nonspecific reaction inhibitor of the invention can sufficiently inhibit a nonspecific reaction in an immunoassay is not clear. However, since the anti-mammal-derived IgM antibody used in the invention shows reactivities with dog IgM, cat IgM and human IgM indicated by the certain absorbance ratios but does not show any strong reactivity with particular IgM, it is believed that the anti-mammal-derived IgM antibody is highly likely to recognize a site common among mammals such as human, dog and cat. Since the nonspecific reaction inhibitor of the invention contains such an anti-mammal-derived IgM antibody, it is speculated that the nonspecific reaction inhibitor acts on various nonspecific factors and strongly inhibits a nonspecific reaction which could not be inhibited by the conventional techniques.

The ratio (A2/A1) of the absorbance A2 which is obtained when the reaction of the anti-mammal-derived IgM antibody of the invention with cat IgM is carried out to the absorbance A1 which is obtained when the reaction of the anti-mammal-derived IgM antibody of the invention with dog IgM is carried out is 0.1 or more and 1.5 or less, in ELISA assay. When the absorbance ratio (A2/A1) is less than 0.1 or exceeds 1.5, the nonspecific reaction cannot be inhibited sufficiently. The upper limit of the absorbance ratio (A2/A1) is preferably 1.0 or less. The lower limit is preferably 0.5 or more. In the invention, the absorbance ratio (A2/A1) is preferably 0.5 or more and 1.5 or less, and more preferably 0.5 or more and 1.0 or less.

The ratio (A3/A1) of the absorbance A3 which is obtained when the reaction of the anti-mammal-derived IgM antibody of the invention with human IgM is carried out to the absorbance A1 which is obtained when the reaction of the anti-mammal-derived IgM antibody of the invention with dog IgM is carried out is 0.5 or more, in ELISA assay. When the absorbance ratio (A3/A1) is less than 0.5, the nonspecific reaction cannot be inhibited sufficiently. The upper limit of the absorbance ratio (A3/A1) is preferably 2.5 or less, and more preferably 1.8 or less. The lower limit is preferably 1.0 or more. In the invention, the absorbance ratio (A3/A1) is preferably 0.5 or more and 2.5 or less, more preferably 0.5 or more and 1.8 or less, and further preferably 1.0 or more and 1.8 or less.

Regarding the anti-mammal-derived IgM antibody used in the invention, it is preferable that the absorbance ratio (A2/A1) is 0.5 or more and 1.5 or less and that the absorbance ratio (A3/A1) is 0.5 or more and 2.5 or less, in ELISA assay. It is more preferable that the absorbance ratio (A2/A1) is 0.5 or more and 1.5 or less and that the absorbance ratio (A3/A1) is 0.5 or more and 1.8 or less. It is further preferable that the absorbance ratio (A2/A1) is 0.5 or more and 1.0 or less and that the absorbance ratio (A3/A1) is 0.5 or more and 1.8 or less. It is particularly preferable that the absorbance ratio (A2/A1) is 0.5 or more and 1.0 or less and that the absorbance ratio (A3/A1) is 1.0 or more and 1.8 or less.

In the invention, the measurement conditions of the ELISA assay for measuring the absorbances A1 to A3 should be the same among the measurements of the absorbances A1 to A3 but are not particularly limited. The absorbances A1 to A3 mean values obtained by subtracting the absorbance measured in a blank (a well in which a color reaction is conducted without the primary antibody but with the secondary antibody) from the absorbances measured when reactions of the anti-mammal-derived IgM antibody with human IgM, dog IgM or cat IgM are carried out, in ELISA assay.

The anti-mammal-derived IgM antibody used in the invention is not particularly limited as long as the anti-mammal-derived IgM antibody shows certain reactivities with human IgM, dog IgM and cat IgM. Such an anti-mammal-derived IgM antibody may be, for example, an antibody obtained using human IgM as an immunogen (which can be called an anti-human IgM antibody), an antibody obtained using dog IgM as an immunogen (which can be called an anti-dog IgM antibody), an antibody obtained using cat IgM as an immunogen (which can be called an anti-cat IgM antibody) or an antibody obtained using IgM derived from a mammal kind other than those above as an immunogen. The anti-mammal-derived IgM antibody is preferably an anti-human IgM antibody, an anti-dog IgM antibody or an anti-cat IgM antibody. Although a class (isotype) of the anti-mammal-derived IgM antibody used in the invention is not particularly limited, either, IgG type is preferable in view of the specificity of the reaction and the easiness of handling.

The anti-mammal-derived IgM antibody used in the invention may be a monoclonal antibody or a polyclonal antibody but is preferably a monoclonal antibody. Moreover, the anti-mammal-derived IgM antibody used in the invention may be a commercial antibody or may be produced as follows according to the need.

When the anti-mammal-derived IgM antibody used in the invention is a monoclonal antibody, after hybridizing spleen cells of a mouse immunized with the immunogen and myeloma cells according to a general method, a hybridoma that produces the target antibody is selected, and the monoclonal antibody produced by the hybridoma can be obtained (for example, see the Köhler and Milstein's technique (Nature 256(1975)495-497)). The method for obtaining the immunogen is not particularly limited, and, for example, a commercial immunogen can be used.

Screening to obtain the hybridoma clone that produces a monoclonal antibody can be conducted by culturing hybridomas for example in a microtiter plate and measuring reactivities of the culture supernatants of wells in which the growth is observed with the immunogen by enzyme immunoassay such as ELISA.

The hybridoma can be cultured using a medium (for example, DMEM containing 10% fetal bovine serum), and a supernatant of a culture solution obtained by centrifugation can be used as a monoclonal antibody solution. Also, ascites can be caused by injecting the hybridoma into the abdominal cavity of the origin animal, and the obtained ascites can be used as a monoclonal antibody solution. The monoclonal antibody is preferably isolated and/or purified.

The applicant has deposited a hybridoma obtained by the method shown in the Examples below (Anti-Dog IgM No. 12), among anti-mammal-derived IgM antibodies used in the invention, at NITE Patent Microorganisms Depositary, National Institute of Technology and Evaluation. The information that specifies the deposition is described below.

The applicant has deposited the hybridoma (Anti-Dog IgM No. 12) under the following conditions.

(1) Name of Depositary Authority: NITE Patent Microorganisms Depositary, National Institute of Technology and Evaluation

(2) Contact: Room 122, 2-5-8 Kazusakamatari, Kisarazu-shi, Chiba 292-0818, Japan, Telephone Number 0438-20-5580

(3) Accession No.: NITE BP-02556

(4) Label for Identification: Anti-Dog IgM No. 12

(5) Date of Original Deposit: Oct. 10, 2017

When the anti-mammal-derived IgM antibody used in the invention is a polyclonal antibody, the antibody can be obtained by separating the target antibody from an antiserum obtained by immunizing an animal for production (for example, mouse, rat, guinea pig, dog, goat, sheep, pig, horse, cattle, human and the like) with the immunogen according to a general method. The method for obtaining the immunogen is not particularly limited, and, for example, a commercial immunogen can be used.

The nonspecific reaction inhibitor containing the anti-mammal-derived IgM antibody may be composed of the anti-mammal-derived IgM antibody only or may contain a component other than the anti-mammal-derived IgM antibody within the scope in which the effects of the invention are not prevented. Moreover, one kind of anti-mammal-derived IgM antibody may be used for the nonspecific reaction inhibitor of the invention, and two or more kinds thereof can also be used in combination. Examples of the other component include buffers such as phosphates and tris(hydroxymethyl)aminomethane, antiseptics such as sodium azide, inorganic salts such as sodium chloride and potassium chloride and the like.

The state of the nonspecific reaction inhibitor of the invention is not particularly limited and may be solid or liquid. In the case of liquid, the nonspecific reaction inhibitor can be prepared by dissolving or suspending the components which the nonspecific reaction inhibitor contains in a solvent. Examples of the solvent include water, organic solvents such as glycerol, mixed solvents thereof and the like.

The anti-mammal-derived IgM antibody content in the nonspecific reaction inhibitor of the invention is not particularly limited and can be appropriately adjusted based on the kind of the analyte, the amount of the analyte, the measurement conditions of the immunoassay and the like. For example, the anti-mammal-derived IgM antibody content in the nonspecific reaction inhibitor used for one analyte is preferably 0.5 μg or more and 20 or less, more preferably 1 μg or more and 15 μg or less, and further preferably 2 μg or more and 10 μg or less.

The immunoassay to which the nonspecific reaction inhibitor of the invention can be applied is not particularly limited as long as the immunoassay is a method for measuring a substance to be detected in an analyte using immunoreaction, and the effects can be exhibited in such a method. Examples include enzyme immunoassay (for example, ELISA), agglutination assay, immunochromatography, radioimmunoassay, nephelometry and the like, and enzyme immunoassay, agglutination assay or immunochromatography is preferable. The nonspecific reaction inhibitor of the invention is particularly useful for immunochromatography, which is considered to be easy to handle, in view of the easiness of collection of the analyte.

Next, the immunochromatographic test strip of the invention is explained. The immunochromatographic test strip of the invention contains the nonspecific reaction inhibitor described above.

The immunochromatographic test strip of the invention can be used, for example, for determining a presence or absence of a substance to be detected or measuring a concentration of the substance to be detected, using immunochromatography.

The immunochromatographic test strip of the invention is not particularly limited except that the nonspecific reaction inhibitor described above is contained, and a structure can be similar to those of known immunochromatographic test strips. In the invention, the nonspecific reaction inhibitor may be contained in a state capable of involving in immunoreaction, in a member in which a liquid phase containing the analyte develops through a capillary phenomenon, of the members constituting the immunochromatographic test strip. In this manner, the immunoreaction can be conducted in the presence of the nonspecific reaction inhibitor, and a nonspecific reaction can be inhibited.

Although the immunochromatographic test strip of an embodiment of the invention is explained below based on the drawings, the immunochromatographic test strip of the invention is not limited to the embodiment below.

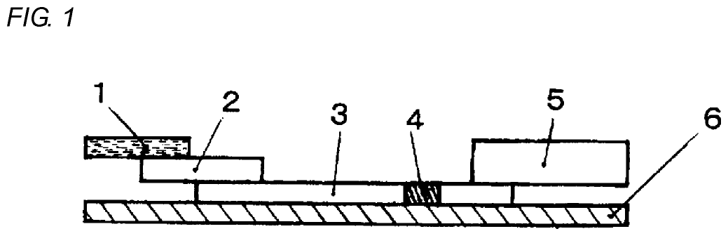

The immunochromatographic test strip of an embodiment of the invention shown in FIG. 1 has a sample pad 1, a conjugate pad 2, a membrane pad 3, an absorption pad 5 and a backing sheet 6.

The sample pad 1 is a member which is provided to apply a sample containing the analyte and to send the sample to a downstream through a capillary phenomenon. A known material can be used as the material of the sample pad 1, and examples include microparticles of a ceramic such as silica, titania, zirconia, ceria and alumina, polyurethane, polyester, polyethylene, polyvinyl chloride, polyvinylidene fluoride, nylon, nitrocellulose, cellulose acetate, glass fibers, cotton and the like. The size and the shape of the sample pad 1 are not particularly limited and should be appropriate in view of the actual operation and the observation of the reaction results. The sample pad 1 may have a function of a conjugate pad described below.

The conjugate pad 2 is a member containing a labeling reagent obtained by labeling an antibody or an antigen which specifically reacts with the substance to be detected in the analyte with a labeling substance. When the sample containing the analyte passes through the conjugate pad 2, a complex of the substance to be detected and the labeling reagent is formed. A known material can be used as the material of the conjugate pad 2, and examples include microparticles of a ceramic such as silica, titania, zirconia, ceria and alumina, polyurethane, polyester, polyethylene, polyvinyl chloride, polyvinylidene fluoride, nylon, nitrocellulose, cellulose acetate, glass fibers, cotton and the like. The size and the shape of the conjugate pad 2 are not particularly limited and should be appropriate in view of the actual operation and the observation of the reaction results.

The labeling substance is not particularly limited, and a known labeling substance such as nanoparticles of a metal such as gold, silver or platinum and latex particles can be used. An average particle diameter of the metal nanoparticles is not particularly limited but is preferably 10 nm to 150 nm, and more preferably 20 nm to 100 nm. The average particle diameter of the latex particles is not particularly limited but is preferably 100 nm to 500 nm, and more preferably 100 nm to 250 nm. Gold nanoparticles are preferably used for the labeling substance, since the presence or absence of the substance to be detected in the analyte can be determined visually.

Regarding the antibody or the antigen in the labeling reagent, a commercial antibody or antigen can be used as long as the antibody or the antigen can specifically bind to the substance to be detected in the analyte, and an antibody or an antigen which is produced can be used according to the need. When the substance to be detected is an antigen, an antibody which can specifically bind to the antigen is preferable. The antibody may be a monoclonal antibody or a polyclonal antibody. Such an antibody can be produced, for example, by a known method by sensitizing an animal by an antigen which is the substance to be detected. Specific examples of the animal kind include mouse, rat, guinea pig, dog, goat, sheep, pig, horse, cattle, human and the like, but the animal kind is not limited to these examples.

The antibody or antigen content of the labeling reagent is not particularly limited but is preferably 0.01 μg or more and 10 μg or less, more preferably 0.02 μg or more and 5 μg or less, and further preferably 0.02 μg or more and 1 μg or less, per one test strip.

The membrane pad 3 is a member having a detection part 4 for determining, for example, the presence or absence of the substance to be detected or measuring a concentration of the substance to be detected, by detecting the substance to be detected. A capturing reagent containing an antibody or an antigen for capturing the substance to be detected is immobilized on the detection part 4. When the substance to be detected is contained in the analyte, a complex of the labeling reagent, the substance to be detected and the capturing reagent is formed, and the detection part 4 develops a color. When the substance to be detected is not contained in the analyte, the complex of the labeling reagent, the substance to be detected and the capturing reagent is not formed, and thus the detection part 4 does not develop a color.

A known material can be used as the material of the membrane pad 3, and examples include microparticles of a ceramic such as silica, titania, zirconia, ceria and alumina, polyurethane, polyester, polyethylene, polyvinyl chloride, polyvinylidene fluoride, nylon, nitrocellulose, cellulose acetate, glass fibers, cotton and the like. The size and the shape of the membrane pad 3 are not particularly limited and should be appropriate in view of the actual operation and the observation of the reaction results.

The antibody or the antigen used for the capturing reagent may be the same antibody or antigen as the antibody or antigen contained in the labeling reagent or a different antibody or antigen.

Regarding the antibody or the antigen used for the capturing reagent, a commercial antibody or antigen can be used as long as the antibody or the antigen can specifically bind to the substance to be detected in the analyte, and an antibody or an antigen which is produced can be used according to the need. When the substance to be detected is an antigen, an antibody which can specifically bind to the antigen is preferable. The antibody may be a monoclonal antibody or a polyclonal antibody. Such an antibody can be produced by a known method by sensitizing an animal by an antigen which is the substance to be detected. Specific examples of the animal kind include mouse, rat, guinea pig, dog, goat, sheep, pig, horse, cattle, human and the like, but the animal kind is not limited to these examples.

The antibody or antigen content used for the capturing reagent is not particularly limited but is preferably 0.1 μg or more and 10 μg or less, more preferably 0.2 μg or more and 5 μg or less, further preferably 0.2 μg or more and 4 μg or less, per one test strip.

The shape of the detection part is not particularly limited, and examples are a line, a circle and the like. A line is preferable in view of the visibility and the detection efficiency.

In order to prevent the deterioration of the analysis accuracy due to nonspecific adsorption, the membrane pad 3 can be subjected to blocking treatment by a known method according to the need. For the blocking treatment, in general, a protein such as bovine serum albumin, skim milk, casein and gelatin is preferably used. After the blocking treatment, the membrane pad 3 may be washed with one or a combination of two or more of surfactants such as Tween (registered trademark) 20, Triton X-100 (registered trademark) and SDS according to the need.

The absorption pad 5 is a member for absorbing the excessive sample or the like which has passed through the membrane pad 3. A known material can be used as the material of the absorption pad, and examples include microparticles of a ceramic such as silica, titania, zirconia, ceria and alumina, polyurethane, polyester, polyethylene, polyvinyl chloride, polyvinylidene fluoride, nylon, nitrocellulose, cellulose acetate, glass fibers, cotton and the like. The size and the shape of the absorption pad 5 are not particularly limited and should be appropriate in view of the actual operation and the observation of the reaction results.

The backing sheet 6 is a member used as a support for fixing the members such as the sample pad 1, the conjugate pad 2, the membrane pad 3 and the absorption pad 5. The material of the backing sheet is not particularly limited, and, for example, various conventionally known materials which become non-permeable or non-breathable with respect to the sample due to an adhesive can be used. The size and the shape of the backing sheet 6 are not particularly limited and should be appropriate in view of the actual operation and the observation of the reaction results.

In the immunochromatographic test strip of an embodiment of the invention, the nonspecific reaction inhibitor is contained in at least one of the sample pad 1, the conjugate pad 2 and the membrane pad 3.

The anti-mammal-derived IgM antibody content of the nonspecific reaction inhibitor contained in the immunochromatographic test strip of the invention is not particularly limited but is preferably 0.5 μg or more and 20 μg or less, more preferably 1 μg or more and 15 μg or less, and further preferably 2 μg or more and 10 μg or less, per one test strip. Within the range, a nonspecific reaction can be inhibited strongly.

Next, the immunochromatographic test kit of the invention is explained.

The test kit in the invention means a kit which includes two or more articles such as a reagent required for the immunoassay. The immunochromatographic test kit of the invention is not particularly limited except that the nonspecific reaction inhibitor described above is contained, and the structure can be similar to those of known immunochromatographic test kits.

In the invention, the nonspecific reaction inhibitor should be contained in the immunochromatographic test kit in a state capable of involving in immunoreaction. For example, the nonspecific reaction inhibitor may be contained individually as a reagent or may be contained in a reagent such as an analyte dilution solution used for the immunoassay or in a test strip or the like.

In an embodiment of the invention, the immunochromatographic test kit has a reagent required for the immunoassay in addition to a test strip. The test strip is not particularly limited, and, for example, a test strip composed of the sample pad, the conjugate pad, the membrane pad, the absorption pad, the backing sheet and the like described above can be used. The reagent required for the immunoassay is not particularly limited, and examples include an analyte dilution solution, a development solution and the like.

In an embodiment of the invention, the nonspecific reaction inhibitor is contained at least in the test strip or the reagent. More specifically, the nonspecific reaction inhibitor is contained in at least one of the sample pad, the conjugate pad, the membrane pad and the reagent. In this manner, the antigen-antibody reaction can be conducted in the presence of the nonspecific reaction inhibitor, and a nonspecific reaction can be inhibited.

The anti-mammal-derived IgM antibody content in the nonspecific reaction inhibitor contained in the immunochromatographic test kit of the invention is not particularly limited but is preferably 0.5 μg or more and 20 μg or less, more preferably 1 μg or more and 15 μg or less, and further preferably 2 μg or more and 10 μg or less, per one test kit. Within the range, a nonspecific reaction can be inhibited efficiently without increasing a viscosity of a solution.

Next, the immunoassay of the invention is explained.

In the immunoassay of the invention, immunoreaction is conducted in the presence of the nonspecific reaction inhibitor described above. By conducting the immunoreaction in the presence of the nonspecific reaction inhibitor, a nonspecific reaction other than the targeted immunoreaction can be inhibited.

The immunoassay of the invention is not particularly limited as long as the immunoassay is a method for quantitatively or qualitatively measuring the substance to be detected in an analyte, using immunoreaction. Examples include enzyme immunoassay (for example, ELISA), agglutination assay, immunochromatography, radioimmunoassay, nephelometry and the like, and enzyme immunoassay, agglutination assay or immunochromatography is preferable. The immunoassay of the invention is particularly useful for immunochromatography, which is considered to be easy to handle, in view of the easiness of collection of the analyte.

The analyte used for the immunoassay of the invention is not particularly limited, and examples include serum, plasma, whole blood, urine, saliva, nasal secretion and the like. The analyte is typically a human-derived analyte.

Examples of the substance to be detected which can be measured by the immunoassay of the invention include viruses, bacteria, parasites, metabolites and the like contained in the analyte and more specifically include proteins, peptides, polysaccharides, complex carbohydrates and the like that these examples have. In particular, an antigen contained in the analyte in a small amount is suitable. This is because, as concentration of the antigen contained in the analyte decreases to a very smaller value, the influence of a nonspecific reaction becomes relatively greater, and the nonspecific reaction inhibitor of the invention is useful.

The immunoreaction in the invention is not particularly limited as long as the immunoreaction is conducted in the presence of the nonspecific reaction inhibitor and can be conducted according to a general method. For example, the immunoreaction can be conducted by bringing the analyte and the nonspecific reaction inhibitor into contact before the immunoreaction and then bringing them into contact with an antibody or an antigen which can bind to the substance to be detected in the analyte. Moreover, the immunoreaction can be conducted by bringing an antibody or an antigen which can bind to the substance to be detected in the analyte and the nonspecific reaction inhibitor into contact before the immunoreaction and then bringing them into contact with the analyte.

The anti-mammal-derived IgM antibody content of the nonspecific reaction inhibitor used in the invention is not particularly limited and can be appropriately adjusted based on a kind of the analyte, an amount of the analyte, measurement conditions of the immunoassay and the like. For example, the anti-mammal-derived IgM antibody content in the nonspecific reaction inhibitor used for one analyte is preferably 0.5 μg or more and 20 μg or less, more preferably 1 μg or more and 15 μg or less, and further preferably 2 μg or more and 10 μg or less.

When the immunoassay of the invention is immunochromatography, an antigen can be detected for example by applying a sample obtained by adding the nonspecific reaction inhibitor in advance to an analyte containing the antigen to a solid phase and forming an immune complex in the solid phase. Moreover, an antigen can be detected by developing an analyte containing the antigen in a solid phase such as a sample pad or a conjugate pad to which the nonspecific reaction inhibitor has been added in advance and forming an immune complex in the solid phase.

When the immunoassay of the invention is enzyme immunoassay (for example, ELISA), a concentration of an antigen can be measured for example by adding the nonspecific inhibitor in advance to an analyte containing the antigen and then conducting an antigen-antibody reaction according to a general method.

### EXAMPLES

Although the invention is explained in detail below based on Examples, the invention is not limited to the Examples below.

### [Anti-Mammal-Derived IgM Antibodies]

Anti-dog IgM antibodies and anti-cat IgM antibodies were produced as follows. Commercial anti-human IgM antibodies were used.

### [Anti-Dog IgM Antibodies]

Monoclonal antibodies of anti-dog IgM antibodies were produced as follows according to a general method using dog IgM (manufactured by Rockland, product name DOG IgM Whole molecule) as an immunogen. The dog IgM in an amount of 100 μg and an equivalent amount of Aduvant Complete Freund (Difco) were mixed, and a mouse (BALB/c, five weeks old, Japan SLC, Inc.) was immunized three times and spleen cells thereof were used for cell fusion. Mouse myeloma cells, Sp2/0-Ag14 cells (Shulman et al., Nature, 276, 269-270, 1978) were used for the cell fusion. A culture medium obtained by adding 0.3 mg/ml L-glutamine, 100 U/ml penicillin G potassium, 100 μg/ml streptomycin sulfate and 40 μg/ml Gentacin to Dulbecco's Modified Eagle Medium (DMEM) (Gibco) and further adding fetal bovine serum (JRH) to be 10% was used for culturing the cells. The cells were fused by mixing the spleen cells of the immunized mouse and Sp2/0-Ag14 cells and adding polyethylene glycol solution (Sigma) thereto. The fused cells were cultured in HAT-DMEM [serum-added DMEM containing 0.1 mM sodium hypoxantine, 0.4 μM aminopterin and 0.016 mM thymidine (Gibco)], and production of antibodies in culture supernatant was confirmed by enzyme immunoassay (ELISA). Antibody production-positive cells were cultured in HT-DMEM [serum-added DMEM containing 0.1 mM sodium hypoxantine and 0.16 mM thymidine] and further cultured in serum-added DMEM.

The cloned cells were injected into the abdominal cavities of mice (BALB/c, retired, Japan SLC, Inc.) to which 2,6,10,14-tetramethylpentadecane (Sigma) had been injected, and the ascites were collected. The ascites were subjected to a protein G column, and monoclonal antibodies were purified. Among the obtained antibodies, five monoclonal antibodies (Nos. 12, 32, 70, 75 and 80) were used for the tests described below. The isotype of the antibodies was IgG type. Among the antibodies, the anti-dog IgM antibody of No. 12 is a monoclonal antibody to dog IgM produced from the hybridoma with the accession No. NITE BP-02556 described above.

### [Anti-Cat IgM Antibodies]

Anti-cat IgM antibodies were produced by the same method as that for producing the anti-dog IgM antibodies except that 100 μg of cat IgM (manufactured by Rockland, product name CAT IgM Whole molecule) was used as the immunogen instead of the dog IgM. Among the obtained antibodies, two monoclonal antibodies (Nos. 1 and 7) were used for the tests described below. The isotype of the antibodies was IgG type.

### [Anti-Human IgM Antibodies]

As anti-human IgM antibodies, commercial products (manufactured by XEMA, product names: X611, X612 and X613) were used for the tests described below. The isotype of the antibodies was IgG type.

### Test Example 1

In Test Example 1, reactivities of the anti-mammal-derived IgM antibodies (the anti-dog IgM antibodies, the anti-human IgM antibodies and the anti-cat IgM antibodies) with dog IgM, cat IgM and human IgM were measured by the ELISA assay below.

### Example 1

The reactivities of the anti-dog IgM antibody (No. 12) with dog IgM, cat IgM and human IgM were measured by the ELISA assay below.

### [ELISA Assay]

First, 100 μL of a solution of 4 ng/mL dog IgM (manufactured by Rockland, product name DOG IgM Whole molecule), cat IgM (manufactured by Rockland, product name CAT IgM Whole molecule) or human IgM (manufactured by Oriental Yeast Co., Ltd., product name human IgM) in 50 mM carbonate buffer of pH 9.5 was put to a Nunc Immuno modules (manufactured by Thermo Fisher Scientific, code 469949) ELISA 96-well plate, and the plate was incubated at 4° C. for 16 hours. After 16 hours, the solution was removed, and the wells were washed three times with 300 μL of PBST (0.05% Tween 20 in PBS). The liquids remaining in the wells were removed by hitting the plate onto a paper towel. As a blocking liquid, 300 μL of 5% BSA (BSA: manufactured by Oriental Yeast Co., Ltd.) in PBST was added, and the plate was incubated at 37° C. for an hour. Then, the BSA solution was removed, and the wells were washed three times with 300 μL of PBST (0.05% Tween 20 in PBS). The liquids remaining in the wells were removed by hitting the plate onto a paper towel.

As the primary antibody, 100 μL of 5 μg/mL anti-dog IgM antibody (No. 12) produced above in 50% blocking solution (a primary antibody solution) was added to the wells, and the plate was incubated at 37° C. for an hour. Then, the primary antibody solution was removed, and the wells were washed three times with 300 μL of PBST (0.05% Tween 20 in PBS).

As the secondary antibody, 100 μL of 1 mg/mL Anti Mouse IgG (H+L), Rabbit, IgG Whole, Peroxidase Cojugated (manufactured by Wako Pure Chemical Industries, Ltd., code 014-17611) was added to the wells, and the plate was incubated at 37° C. for 1.5 hours. Then, the BSA solution was removed, and the wells were washed three times with 300 μL of PBST (0.05% Tween 20 in PBS). The liquids remaining in the wells were removed by hitting the plate onto a paper towel.

Sure Blue Reserve TMB Microwell Peroxidase Substrate (1-Component) (manufactured by KPL, code 53-00-01) in an amount of 100 μL was added to the wells as a chromogenic substrate, and a reaction was carried out for 15 minutes. The reaction was stopped by adding 100 μL of 2N sulfuric acid. Then, the absorbances at 450 nm were measured using a microplate reader (manufactured by BIORAD).

Moreover, an absorbance of a blank (a well in which the color reaction was conducted without the primary antibody but with the secondary antibody) was measured, and the absorbances A1 to A3 were determined by subtracting the absorbance from the absorbances measured above. The results are shown in Table 1.

### Examples 2 and 3

ELISA assay was conducted in the same manner as in Example 1 except that anti-dog IgM antibodies No. 32 and No. 75 produced above were used instead of anti-dog IgM antibody No. 12 as the primary antibody. The results are shown in Table 1.

### Examples 4 to 6

ELISA assay was conducted in the same manner as in Example 1 except that commercial anti-human IgM antibodies X611 to X613 (manufactured by XEMA) were used instead of anti-dog IgM antibody No. 12 as the primary antibody. The results are shown in Table 1.

### Comparative Examples 1 and 2

ELISA assay was conducted in the same manner as in Example 1 except that anti-dog IgM antibodies No. 70 and No. 80 produced above were used instead of anti-dog IgM antibody No. 12 as the primary antibody. The results are shown in Table 1.

### Comparative Examples 3 and 4

ELISA assay was conducted in the same manner as in Example 1 except that anti-cat IgM antibodies No. 1 and No. 7 produced above were used instead of anti-dog IgM antibody No. 12 as the primary antibody. The results are shown in Table 1 below.

TABLE 1
(Abs)

| | | | | | | | | Compar- | Compar- | Compar- | Compar- |
|---|---|---|---|---|---|---|---|---|---|---|---|
| | | | | | | | | ative | ative | ative | ative |
| | | Exam- | Exam- | Exam- | Exam- | Exam- | Exam- | Exam- | Exam- | Exam- | Exam- |
| | | ple 1 | ple 2 | ple 3 | ple 4 | ple 5 | ple 6 | ple 1 | ple 2 | ple 3 | ple 4 |
| | | Anti-dog IgM antibody | | | Anti-human IgM antibody | | | Anti-dog IgM antibody | | Anti-cat IgM antibody | |
| | | No. 12 | No. 32 | No. 75 | X611 | X612 | X613 | No. 70 | No. 80 | No. 1 | No. 7 |
| Antigen | Dog IgM | 0.695 | 0.593 | 0.629 | 0.492 | 0.204 | 0.243 | 0.889 | 1.083 | 0.35 | 0.37 |
| | (absorbance A1) | | | | | | | | | | |
| | Cat IgM | 0.432 | 0.504 | 0.413 | 0.439 | 0.04 | 0.077 | 0.325 | 0.373 | 0.826 | 0.65 |
| | (absorbance A2) | | | | | | | | | | |
| | Human IgM | 0.549 | 0.591 | 0.499 | 0.72 | 0.465 | 0.482 | 0.328 | 0.464 | 0.182 | 0.529 |
| | (absorbance A3) | | | | | | | | | | |
| Absorbance | A2/A1 | 0.622 | 0.850 | 0.657 | 0.892 | 0.196 | 0.317 | 0.366 | 0.344 | 2.360 | 1.757 |
| ratio | A3/A1 | 0.790 | 0.997 | 0.793 | 1.463 | 2.279 | 1.984 | 0.369 | 0.428 | 0.520 | 1.430 |

### Test Example 2

In Test Example 2, using human serum showing nonspecific reactions as the analyte, the inhibitory effects of the anti-IgM antibodies of the Examples and the Comparative Examples, which were examined for their reactivities with dog IgM, cat IgM and human IgM in Test Example 1, and a conventionally known heterophilic blocking reagent HBR on nonspecific reactions in an immunoassay were evaluated.

Specifically, test strips having a membrane pad 3 having a detection part 4, a sample pad 1, a conjugate pad 2 and an absorption pad 5 on a backing sheet 6 as shown in FIG. 1 and development samples were produced as follows, and measurement was conducted by immunochromatography. The inhibitory effects on nonspecific reactions were evaluated.

### (1) Production of Sample Pad

A nonwoven cloth composed of glass fibers (manufactured by Millipore Corporation: 300 mm×30 mm) was used as the sample pad.

### (2) Production of Conjugate Pad

To 0.5 ml of a colloidal gold suspension (manufactured by Tanaka Kikinzoku Kogyo K.K.: LC 40 nm), 0.1 ml of anti-Zika virus NS1 antibody (manufactured by Aaltobioreagent, product name Anti-Zika virus NS1 Antibody) which had been diluted to a concentration of 0.05 mg/ml with a phosphate buffer (pH 7.4) was added, and the mixture was left to stand still at room temperature for 10 minutes.

Next, 0.1 ml of a phosphate buffer (pH 7.4) containing 1 mass % bovine serum albumin (BSA) was added, and the mixture was further left to stand still at room temperature for 10 minutes. Then, after stirring thoroughly, the mixture was centrifuged at 8000×g for 15 minutes, and the supernatant was removed. Then, 0.1 ml of a phosphate buffer (pH 7.4) containing 1 mass % BSA was added. A labeling reagent was produced by the above procedures.

A solution obtained by adding 300 μL of a 10 mass % aqueous trehalose solution and 1.8 mL of distilled water to 300 μL of the labeling reagent produced above was evenly applied to a 12 mm×300 mm glass fiber pad (manufactured by Millipore Corporation) and then dried with a vacuum dryer, and the conjugate pad was thus produced.

### (3) Production of Detection Part

A sheet composed of nitrocellulose (manufactured by Millipore Corporation, product name: HF120, 300 mm×25 mm) was used as a membrane.

Next, 150 μL of a solution obtained by diluting anti-Zika virus NS1 antibody (manufactured by Aaltobioreagent, product name Anti-Zika virus NS1 Antibody) to a concentration of 1.0 mg/ml with a phosphate buffer (pH 7.4) containing 5 mass % isopropyl alcohol was applied to a detection part on the dried membrane in a line with a width of 1 mm using a dispenser “XYZ3050” (manufactured by BIODOT) for immunochromatography at an amount of 1 μL/mm (25 μL per sheet).

Moreover, to check whether the gold nanoparticle labeling reagent has been developed or not and to check the development speed, a solution obtained by diluting goat-derived antiserum having a broad affinity range with the gold nanoparticle labeling substance with a phosphate buffer (pH 7.4) was applied to a control part (a control line) in the downstream of the detection part. Then, the membrane was dried at 50° C. for 30 minutes and dried at room temperature overnight.

### (4) Production of Test Strip

The sample pad, the conjugate pad and an absorption pad composed of a nonwoven cloth made of glass fibers were attached one by one to the membrane pad having the detection part. Then, the obtained product was cut with a width of 5 mm with a cutter and attached onto a backing sheet (manufactured by Kuramoto Sangyo Co., product name backing sheet for immunochromatography), and test strips were thus obtained. The length of the conjugate pad in the direction of sample development was adjusted to 12 mm.

### (5) Preparation of Development Samples

Human serum showing nonspecific reactions (manufactured by SCIPAC, product name Normal Female Serum) was used as the analyte. The development samples were prepared by mixing 30 μL of the analyte, 60 μL of a PBS solution containing 0.5% Triton X-100 and 4 μg of any of the anti-IgM antibodies of Examples 1 to 6 and Comparative Examples 1 to 4 used in Test Example 1 or 4 μg of HBR (manufactured by Scantibody). A control was prepared using 10 μL of PBS instead of the anti-IgM antibodies.

### (6) Measurement

Using the test strips and the development samples produced above, the inhibitory effects on nonspecific reactions were tested by the following method.

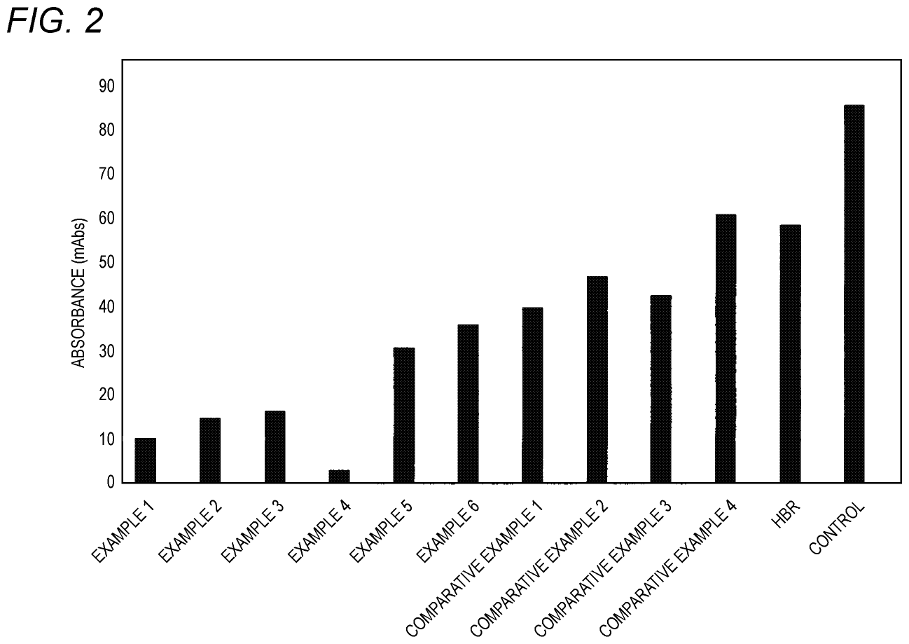

The development samples each in an amount of 150 μL were applied to the sample pads of the test strips and developed, and the color strengths of the detection parts (absorbances at 450 nm) were measured after 15 minutes with an immunochromatographic reader. The results are shown in Table 2 and FIG. 2.

TABLE 2
(mAbs)

| | Example 1 | Example 2 | Example 3 | Example 4 | Example 5 | Example 6 |
|---|---|---|---|---|---|---|
| Color | 10.1 | 14.7 | 16.3 | 2.9 | 30.6 | 36 |
| Strength | | | | | | |
| | Comparative | Comparative | Comparative | Comparative | | |
| | Example 1 | Example 2 | Example 3 | Example 4 | HBR | Control |
| Color | 39.7 | 46.8 | 42.6 | 60.7 | 58.4 | 85.4 |
| Strength | | | | | | |

As seen also from the results of the control, it can be understood that the human serum (manufactured by SCIPAC, product name Normal Female Serum) used as the analyte is an analyte causing nonspecific reactions. The Examples using the antibodies of the invention could significantly inhibit the nonspecific reactions also in comparison with the conventionally known heterophilic blocking reagent HBR.

### Test Example 3

In this test, using human serum showing nonspecific reactions (manufactured by SCIPAC, product name Normal Female Serum) as the analyte, the inhibitory effects of anti-human IgG antibody clones on nonspecific reactions were evaluated in the same manner as in Test Example 2.

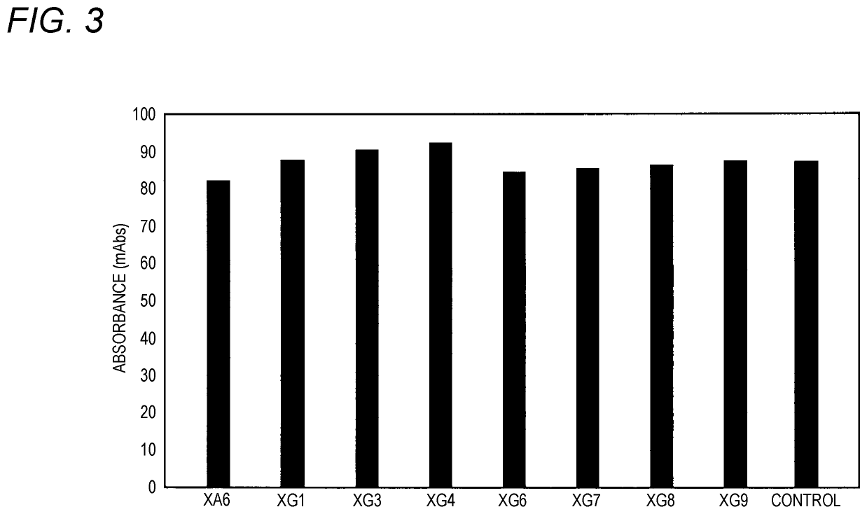

Test strips and development samples were produced and measurement was conducted in the same manners as in Test Example 2 except that anti-human IgG antibody clones (manufactured by XEMA, product names XA6, XG1, XG3, XG4 and XG6 to 9) were used instead of the anti-IgM antibodies of Test Example 2. The results are shown in Table 3 and FIG. 3.

TABLE 3
(mAbs)

| Antibody | XA6 | XG1 | XG3 | XG4 | XG6 | XG7 | XG8 | XG9 | Control |
|---|---|---|---|---|---|---|---|---|---|
| Color | 82.3 | 87.9 | 90.5 | 92.3 | 84.6 | 85.5 | 86.5 | 87.5 | 87.3 |
| Strength | | | | | | | | | |

As seen from the results above, effects of inhibiting the nonspecific reactions could not be obtained even when the anti-human IgG antibody clones were used.

### Test Example 4

In this test, using human serum showing nonspecific reactions (manufactured by SCIPAC, product name Normal Female Serum) as the analyte, the inhibitory effects of IgG or IgY of animal kinds (mouse, rabbit, goat and chicken) on nonspecific reactions were evaluated in the same manner as in Test Example 2.

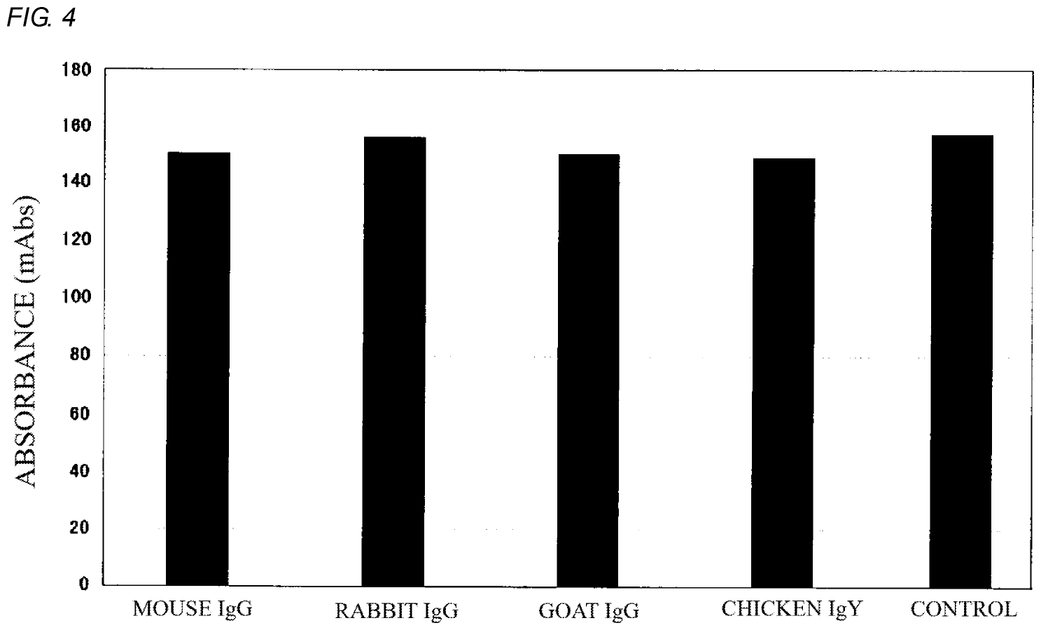

Test strips and development samples were produced and measurement was conducted in the same manners as in Test Example 2 except that the following IgG or IgY of animal kinds (mouse, rabbit, goat and chicken) were used instead of the anti-IgM antibodies used for the development samples in Test Example 2. The results are shown in Table 4 and FIG. 4.

⋅ mouse IgG (manufactured by Nippon Bio-Test Laboratories Inc., product name: purified mouse IgG) ⋅ rabbit IgG (manufactured by Nippon Bio-Test Laboratories Inc., product name: purified rabbit IgG) ⋅ goat IgG (manufactured by Nippon Bio-Test Laboratories Inc., product name: purified goat IgG) ⋅ chicken IgY (manufactured by Nippon Bio-Test Laboratories Inc., product name: purified chicken IgY)

TABLE 4

(mAbs)

| | Mouse | Rabbit | Goat | Chicken | |
|---|---|---|---|---|---|
| Antibody | IgG | IgG | IgG | IgY | Control |
| Color | 150.5 | 156.2 | 150.3 | 149.3 | 157.5 |
| Strength | | | | | |

As seen from the results above, effects of inhibiting the nonspecific reactions could not be obtained even when the IgG or IgY derived from the animal kinds (mouse, rabbit, goat and chicken) were used.

Although the invention has been explained in detail using specific embodiments, it is obvious to one skilled in the art that various changes and modifications can be made without departing from the intension and the scope of the invention. The present application is based on a Japanese patent application filed on May 2, 2017 (patent application No. 2017-091858), which is hereby incorporated by reference in its entirety.

### REFERENCE SIGNS LIST

1. Sample pad
2. Conjugate pad
3. Membrane pad
4. Detection part
5. Absorption pad
6. Backing sheet

1. A nonspecific reaction inhibitor for an immunoassay, comprising an anti-mammal-derived IgM antibody,
in which, in ELISA assay, a ratio (A2/A1) of an absorbance A2 which is obtained when a reaction of the anti-mammal-derived IgM antibody with cat IgM is carried out to an absorbance A1 which is obtained when a reaction of the anti-mammal-derived IgM antibody with dog IgM is carried out is 0.1 or more and 1.5 or less and in which, in ELISA assay, a ratio (A3/A1) of an absorbance A3 which is obtained when a reaction of the anti-mammal-derived IgM antibody with human IgM is carried out to the absorbance A1 which is obtained when the reaction of the anti-mammal-derived IgM antibody with dog IgM is carried out is 0.5 or more.

2. The nonspecific reaction inhibitor according to claim 1, wherein the ratio A3/A1 is 2.5 or less.

3. The nonspecific reaction inhibitor according to claim 1, wherein the anti-mammal-derived IgM antibody is an anti-human IgM antibody, an anti-dog IgM antibody or an anti-cat IgM antibody.

4. The nonspecific reaction inhibitor according to claim 1, wherein the anti-mammal-derived IgM antibody content is 0.5 μg or more and 20 μg or less.

5. The nonspecific reaction inhibitor according to claim 1, wherein the immunoassay is immunochromatography.

6. An immunochromatographic test strip comprising the nonspecific reaction inhibitor according to claim 1.

7. An immunochromatographic test kit comprising the nonspecific reaction inhibitor according to claim 1.

8. An immunoassay for specifically detecting a substance to be detected in an analyte, wherein the immunoreaction is conducted in the presence of the nonspecific reaction inhibitor according to claim 1.

9. The immunoassay according to claim 8 which is enzyme immunoassay, agglutination assay or immunochromatography.

10. The immunoassay according to claim 8 which is immunochromatography.
